(19) United States
(12) Patent Application Publication
(10) Pub. No.: US 2002/0023640 A1
(43) Pub. Date: Feb. 28, 2002

(54) Respiratory apparatus including liquid ventilator

(76) Inventor: Chris Nightengale, Englewood, CO (US)

Correspondence Address: Brent P. Johnson, SHERIDAN ROSS P.C., Suite 1200, 1560 Broadway, Denver, CO, 80202-5141, US

(21) Appl. No.: 09/855,225

(22) Filed: May 14, 2001

Related U.S. Application Data
(60) Provisional application No. 60/203,662, filed on May 12, 2000.

(51) Int. Cl. A62B 7/00; A62B 9/00; A61M 15/00; A61M 16/00; A62B 18/00; F24F 5/00; F24J 3/00

(52) U.S. Cl. 128/200.24; 128/204.15; 128/204.17; 128/913

(57) ABSTRACT

A respiratory apparatus is provided including a liquid ventilator. The primary components of the apparatus include a ventilator, control valve, and chiller/oxygenator. The invention utilizes a closed loop circulation of a breathable liquid to provide liquid ventilation and pulmonary cooling. The ventilator is a dual-chambered device which may be hand or machine actuated. A dual compression force is applied to the ventilator which induces a patient's inspiration of oxygenated and cooled liquid from an inspiration chamber, and simultaneously induces transfer of oxygen depleted, heated liquid into the oxygenator/chiller from an expiration chamber. Upon release of the compression force, the ventilator returns to its normally open or uncompressed state inducing evacuation of the liquid from the patient's lungs into the expiration chamber and flow of re-oxygenated and chilled liquid into the inspiration chamber of the ventilator, thus readying the ventilator for the next inspiration phase.

### CROSS-REFERENCE TO RELATED APPLICATIONS

Priority is claimed from U.S. Provisional Patent Application No. 60/203,662, filed on May 12, 2000 entitled “Method and Apparatus For Providing Lung, Heart and Brain Protection During Cardiac Arrest”, which is incorporated by reference in its entirety.

### FIELD OF THE INVENTION

The present invention relates generally to respiratory apparatuses, and more particularly, to a liquid ventilator for pulmonary ventilation and cooling.

### BACKGROUND OF TE INVENTION

Pulmonary ventilation utilizing breathable liquids has been the subject of a number of prior art inventions. Generally, each of these inventions relate to a device or system for pulmonary ventilation via a breathable liquid which provides adequate oxygenation and the elimination of carbon dioxide during the ventilation process. One presently preferred type of breathable liquid is perfluorocarbon liquids. Perfluorocarbon liquids are also referred to as perfluorocarbons or simplyPFCs, and are derived from common organic compounds in which fluorine atoms have replaced carbon bound hydrogen atoms. PFCs are colorless, odorless, nonflammable liquids which have a high dielectric strength and resistivity. They are substantially insoluble in water, are denser than water, exhibit low surface tensions, and have low viscosities. PFCs have high affinity for gases, having the capability to dissolve gas at much higher amounts than water. Perfluorocarbons, in addition to having the ability to carry large amounts of oxygen in simple solution, also have excellent heat transfer properties. Thus, perfluorocarbons can be used to establish a temperature gradient across the pulmonary surface. Perfluorocarbon liquids also have utility with respect to artificial blood substitutes, and other treatments such as convective lung hypothermia and lung lavage.

One example of a prior art reference disclosing a liquid ventilator system includes the U.S. Pat. No. 5,706,830. This reference specifically discloses a liquid ventilator system including an inspiratory conduit, a bifurcated bronchial tube, and a pump. The bifurcated bronchial tube has a left lumen for directing flow of oxygen as liquid into the left primary bronchus of a subject, and a right lumen for directing the flow of oxygen as liquid into the right primary bronchus of a subject. A membrane oxygenator and heat exchanger are also provided in the system for oxygenization and temperature control of the perfluorocarbon.

Another example of a prior art reference includes the U. S. Pat. No. 5,492,109. This reference discloses a closed loop, single path circuit for liquid ventilation. The primary components include a reservoir, a pump, an oxygenator, a heat exchanger, an endotracheal tube, a Y connector, a valve and a controller. To simulate inspiration, the controller closes the valve, which is located downstream of the Y connecter, thereby forcing the liquid into the lungs of the patient. After a volume of liquid has been perfused into the patient's lungs, the controller opens the valve. The Y connector is shaped such that the flow of liquid through its inspiratorial limb will cause the liquid in the patient's lungs to be entrained into the flow of the Y connector. In this manner, the Y connector operates as an ejector pump and allows the apparatus to continuously perfuse the liquid through the single path circuit.

Yet another example of a prior art reference includes U.S. Pat. No. 6,105,572. This reference discloses a liquid ventilator system having a dual loop, single pump configuration. The liquid ventilator utilizes two 3-way valves for routing liquid from a reservoir in a regeneration apparatus and to a patient's pulmonary system, and back to the reservoir again. A controller controls the configuration of the 3-way valves and the pump speed of the motor to allow user control over the parameters of inspiration and expiration.

Although the aforesaid ventilator systems may be adequate for their intended purposes, some distinctive disadvantages of these systems are that they are structurally complex, non-portable, and are intended for use only in a hospital setting which can support the ventilator systems. Thus, these systems are not intended for use in emergency treatment.

The great majority of naturally occurring sudden deaths are caused by cardiac disease. Because of the suddenness in which cardiac arrest can occur, emergency medical personnel are often unable to timely respond to cardiac arrest. Response time is of the essence in treating cardiac arrest. Even in those circumstances in which emergency medical personnel are able to initiate timely emergency treatment of a patient, current techniques for treating cardiac arrest are not always adequate.

Most cardiac arrests occur with the onset or worsening of coronary artery obstruction. Animal studies have shown hypothermia to be effective in reducing the extent of tissue injury due to low perfusion, ischemia, and hypoxia. Lowering brain temperature a few degrees Celsius substantially reduces histopathologic tissue injury in laboratory models of neurologic ischemia and cardiac arrest. Resuscitation using a hypothermic cardiac arrest model in dogs cooled by peritoneal lavage shows improved survival and decreased neurologic injury. Hypothermia is used clinically to provide neuroprotection during cardiac surgery requiring circulatory arrest. Studies have shown complete cellular and neurologic recovery using profound hypothermia (18° C.) with circulatory arrest for up to 90 minutes in pediatric cardiac surgery. Cellular metabolism decreases exponentially as core temperature decreases extending the duration that tissue can tolerate ischemia without injury.

Providing rapid hypothermia to the brain, heart, and viscera of an arrest victim while maintaining ventilation and minimal circulation out of the operating room is a challenge Cardiopulmonary bypass cooling and the application of epicardial slush provide hypothermia in cardiac surgery. Heat transfer for core cooling has been evaluated intraoperatively using healthy volunteers.

Experimentally, peritoneal lavage has been shown to decrease core temperature 5°-10° C./h. Heat transfer during surface cooling has also been evaluated using cutaneous thermal flux transducers. Whole body immersion in ice-water slurry leads to approximately 600-800 W-heat loss and decreases core temperature 9°-10° C./h. Finally, four liters of icecold fluid administered intravenously corresponds to a heat transfer of 150 W and 2° C. decrease in core temperature when given over a 1-h period. None of these methods are effective in cooling a cardiac arrest victim because blood flow is primarily directed to the lungs, heart and brain leading to a slow rate of core to surface heat loss. Studies show that peripheral vasoconstriction as with cardiac arrest prevents convective heat loss from the core compartment. The lungs provide a substantial conductive surface for core heat loss.

Heat loss from the lungs by instilling and ventilating cold perfluorocarbon to create a heat sink directly to the core compartment has many advantages. Creating core convective heat loss from the brain and viscera to the lungs requires blood flow from the pulmonary circulation, through the heart, and to the brain and viscera. The endocardium and myocardium are potentially very rapidly cooled because of the immediate circulation of cold blood from the pulmonary circulation and the short distance between the cold perfluorocarbon filled lungs and the epicardial surface. Effective cardiopulmonary resuscitation (CPR) delivers 200-500 cc (or 10-25 cc/100 gm) of blood per minute to the brain. By comparison, global cerebral blood flow averages 50-55 ml/100 gm minute in the awake state. The brain's high metabolic rate and high blood flow requirements lead to rapid injury during arrest, but also offers an opportunity for circulatory cooling as most blood flow is directed to the brain during arrest.

Convective heat loss from sites distant to the lungs depends on the total per minute perfusion and the temperature difference between the perfusing blood and surrounding tissue. Therefore, brain cooling during cardiac arrest depends on maximizing the cardiac output with CPR and maximizing the temperature gradient between blood and tissue. Within limits, compensatory mechanisms attempt to protect the central nervous system after cardiac arrest. The cerebral circulation is extremely sensitive to changes in the partial pressure of carbon dioxide. Within the physiologic range, a 1-mm Hg change in PaCO₂ results in a 3-4% change in cerebral blood flow. Cerebral blood flow increases by a factor of 4 as arterial PaCO₂ doubles. Cerebral cooling during cardiopulmonary bypass that allows increased PaCO₂ shows improved brain cooling efficiency and more even cooling due to maximally distributed cerebral blood flow. This same mechanism is intact during cardiac arrest and tends to maximize perfusion from CPR into the cerebral circulation. Maintaining a maximum temperature gradient between blood and tissue depends on continually removing heat from the pulmonary circulation into the pulmonary perfluorocarbon.

From the foregoing, it is apparent that liquid ventilation utilizing perfluorocarbons for a patient undergoing cardiac arrest is potentially highly advantageous. However, for effective initial emergency treatment, the respiratory apparatus used to deliver the flow of perfluorocarbons must be of a quite distinct construction as compared to the prior art discussed above. That is, a simple yet effective respiratory apparatus is needed which can be immediately employed by emergency medical personnel. The apparatus should be portable for use with emergency medical equipment carried by emergency medical personnel. The apparatus should have the capability to be manually operated, and also mechanically operated once the patient has been transported to an emergency room.

### SUMMARY OF THE INVENTION

In accordance with this invention, a portable and disposable respiratory apparatus is provided which provides continuous ventilation of cooled and oxygenated perfluorocarbon into the lungs, and also provides for closed loop recirculation of oxygen depleted and heated perfluorocarbons. The closed loop construction allows the perfluorocarbon to be rechilled, reoxygenated, and recirculated through the lungs. This closed circuit design also minimizes the amount of perfluorocarbon required and potentially allows the perfluorocarbon to be saved and recycled to the manufacture after resuscitation.

As stated above, the respiratory apparatus of this invention is intended to deliver perfluorocarbons for purposes as both an oxygen carrier and a heat transfer agent across the pulmonary surface. The provision of cold perfluorocarbon to provide rapid pulmonary circulation cooling can provide protective hypothermia for cardiac arrest. Further, the respiratory apparatus of this invention provides effective neuro protective and systemic protective hypothermia as an advance in cardiopulmonary resuscitation and life support.

In its simplest form, the respiratory apparatus of this invention includes a ventilator for providing a flow of liquid perfluorocarbon to and from the patient's lungs, and an externally mounted oxygenator and chiller or heat exchanger which reoxygenates and chills the perfluorocarbon. The apparatus can be described as closed loop in that the perfluorocarbon is continually recycled in the respiratory process, without the need for a separate oxygenation/temperature control loop, and also without the need for a separate loop or system for introduction of additional or replacement perfluorocarbon.

The ventilator is characterized by a dual chambered device which provides the force or power for circulation of perfluorocarbons in the patient's lungs to include both active inspiration and active expiration. The ventilator has a pair of movable exterior walls which are simultaneously actuated to provide flow of the breathable liquid through the closed loop system. These movable exterior walls may be hand actuated, or may be actuated by a mechanical device. The chambers of the ventilator include a single expiratory reservoir, and a single inspiratory reservoir. Both reservoirs are of an elongate, symmetrical shape encouraging steady laminar flow of liquid. In the preferred embodiment, the ventilator resembles a bellows structure in that the movable exterior walls are hinged at one end, and are rotatable about the hinge points based upon external force applied to the exterior walls. The ends of the movable exterior walls opposite the hinge points communicate with corresponding biased actuating means which bias or hold the exterior walls in a normally open position maximizing the internal volume of the reservoirs. These biased actuating means can be in the form of a curved shaped memory compliant material, such as plastic. External force or compression overcomes the biased actuating means and, accordingly, reduces the volume of the chambers. A single non-compliant or stiff wall separates the expiratory reservoir from the inspiratory reservoir, thus simplifying the construction of the ventilator.

The ventilator may be sized to fit within the hand of a user. To actuate the exterior walls, the user simply squeezes or compresses the exterior walls together, and repeats this compression or squeezing action to match the desired respiratory rate of the patient. The reservoirs are of symmetrical construction, and have substantially the same internal volume to ensure consistent and equal flow rates of liquid during patent inspiration and expiration. Preferably, the volume of each chamber is approximately 300 cc.

An endotracheal tube communicates with an inlet of the expiratory reservoir and an outlet of the inspiratory reservoir. These inlets and outlets are co-located at the same end of the ventilator. The opposite or opposing end of the ventilator includes an expiratory reservoir outlet and inspiratory reservoir inlet. The oxygenator/heat exchanger is positioned in line between this outlet and inlet for reoxygenating the oxygen depleted liquid and to provide temperature control for the perfluorocarbon liquid. A control valve is placed in line between the endotracheal tube and the ventilator. This control valve can be of a simple toggle valve construction whereby the valve is biased to close the outlet of the inspiratory reservoir, and then is moved to close the inlet of the expiratory reservoir once fluid is forced through the ventilator by application of external force on the exterior walls.

The movable exterior walls, as described above, can be hand actuated. Alternatively, the ventilator may be actuated by a mechanical device which supplies simultaneous pressure to both of the exterior walls in the same manner as hand actuation. The ventilator could be secured by means of a clamping device, and then a pair of opposing force transferring members could be placed adjacent the exterior walls for applying force thereto. The pressure applying members could include a pair of opposed pistons which reciprocate in a back and forth motion to apply and release force against the exterior walls. These pistons could be controlled by a timing device which would be set to match the desired respiration rate of the patient.

Other advantages and benefits of the present invention will be apparent from the following description of the preferred embodiment, taken in conjunction with the accompanying drawings.

### BRIEF DESCRIPTION OF THE DRAWINGS

FIG. 1 is a simplified schematic diagram of the respiratory apparatus of this invention;

FIG. 2 is a perspective view of the respiratory apparatus of this invention, illustrating the primary or basic components making up the apparatus;

FIG. 3 is a schematic cross-sectional diagram illustrating the respiratory apparatus of the invention, and illustrating the flow of liquid through the apparatus, the ventilator being shown in the non-actuated or open position;

FIG. 4 is another schematic cross-sectional diagram of the respiratory apparatus of this invention, illustrating a compression force which has been applied to the ventilator, the ventilator thus being in the actuated or compressed position;

FIG. 5 is a schematic diagram of the control valve which controls flow between the endotracheal tube and the reservoirs of the ventilator; and

FIG. 6 is a schematic diagram of the ventilator along with an external force applying device which can be used to actuate the ventilator.

### DETAILED DESCRIPTION OF THE DRAWINGS

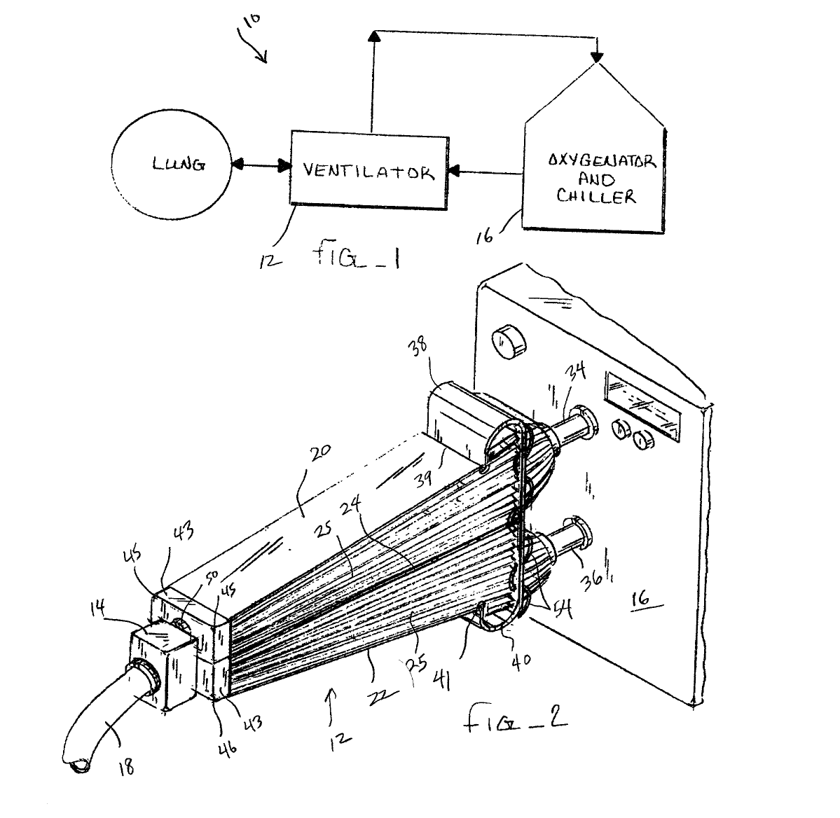

FIG. 1 illustrates a simplified schematic representation of the present invention. The respiratory apparatus 10 includes a ventilator 12 which circulates liquid to and from the lungs of a patient, and fluid to and from an oxygenator and chiller 16 which reoxygenates and cools the liquid. During forced patient inspiration, the ventilator provides a volumetric controlled amount of oxygenated fluid to the patient's lungs, preferably in the form of perfluorocarbon, and wherein the fluid is at a desired temperature. Upon expiration by the patient, fluid is withdrawn from the lungs by the ventilator, and the oxygen depleted fluid is then transferred to the oxygenator and chiller 16 for conditioning. The apparatus provides a closed loop circulation of liquid to and from the patient. Oxygenator/chiller 16 can be a combined unit comprising two commercially available devices which are placed in a single housing. For example, the oxygenator portion could be a Cobe bubble oxygenator made by Cobe Laboratories of Lakewood, Colo., and the heat exchanger portion could be a Cincinnati Sub-Zero cooler/heater made by Cincinnati Sub-Zero Products, Inc. of Cincinnati, Ohio. The combined unit would be portable for use in emergency treatment. Although specific commercial devices have been suggested, it shall be understood that other commercially available devices could be used for the oxygenator/chiller 16.

FIG. 2 illustrates a preferred embodiment of this invention. As shown, one end of the ventilator 12 communicates with the oxygenator and chiller 16, and the opposing end communicates with an endotracheal tube 18 via a control valve 14. Ventilator 12 is characterized by a first movable exterior wall 20, a second movable exterior wall 22, and an internal separating wall 24 positioned between the pair of exterior walls. Walls 20, 22 and 24 are of rigid construction and can be made of a plastic composition. The longitudinal sides of the ventilator 12 are closed by flexible or compliant material 25. Accordingly, the ventilator is separated into two chambers or compartments, namely, an expiratory chamber or reservoir, and an inspiratory chamber or reservoir.

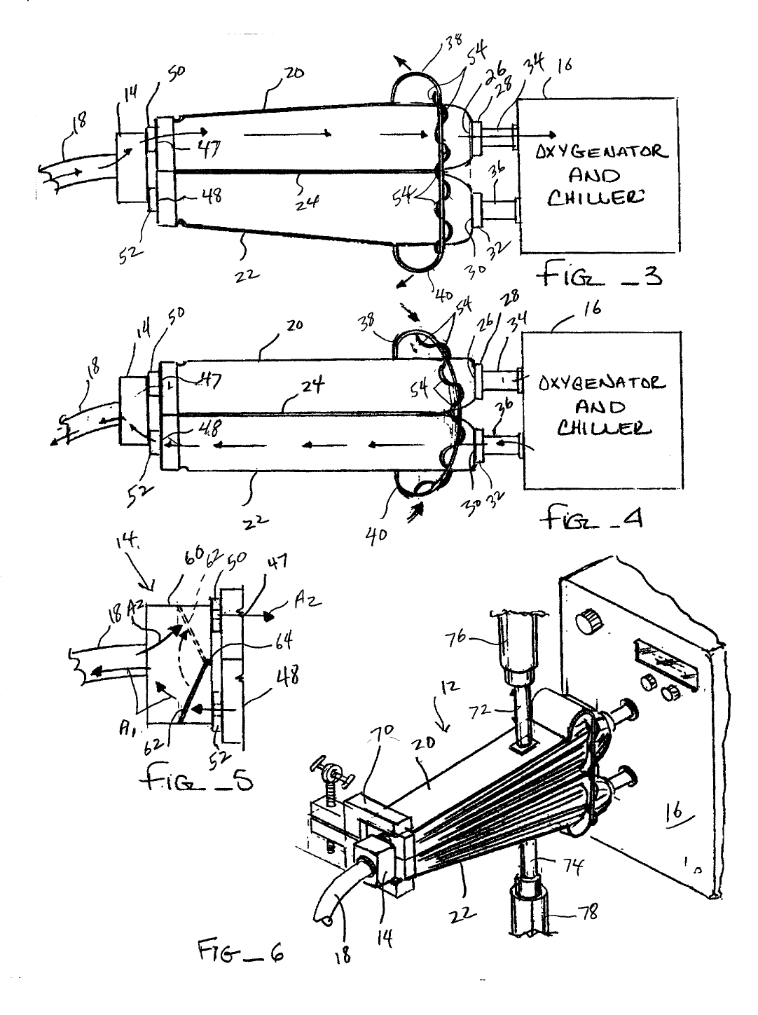

Now also referring to FIGS. 3 and 4, it is seen that the expiratory reservoir is defined as the chamber or space between the first movable exterior wall 20 and one side of the internal separating wall 24. The inspiratory reservoir is defined by the chamber or space between second movable exterior wall 22 and the opposite side of internal separating wall 24. The expiratory reservoir includes an outlet 26. A check valve 28 is mounted to the expiratory reservoir and communicates with the outlet 26. The inspiratory reservoir includes an inlet 30, and a corresponding check valve 32 communicating with the inlet 30. Tube 34 connects to check valve 28 and allows flow of oxygen depleted perfluorocarbon into oxygenator/chiller 16. Tube 36 connects to check valve 30 and allows oxygen enriched and cooled perfluorocarbon to flow into the inspiratory reservoir. From the view of FIGS. 3 and 4, circulation of fluid is clockwise. Check valves 28 and 32 prevent counter-current flow, i.e., counterclockwise circulation. A first biased actuating means 38 connects transversely along edge 39 of exterior wall 20. Similarly, a second biased actuating means 40 connects along the transverse edge 41 of second exterior wall 22. Collectively, actuating means 38 and 40 may form an integral, reverse c-shaped channel member. Actuating means 38 and 40 are biased such that they remain in a spread relationship with one another. That is, the actuating means 38 and 40 prevent walls 20 and 22 from collapsing towards one another without some compression force being applied to walls 20 and 22. The actuating means 38 and 40 can also be constructed of a compliant material, such as plastic. In addition to actuating means 38 and 40, a woven spring 54 can be used to enhance the spring or biased characteristic of the actuating means. As shown in FIG. 2, spring 54 may be traversed in a weave fashion through openings in the longitudinal edge of the actuating means. As the ventilator is compressed, the spring 54 provides an increased biasing force resisting compression.

A first hinge 42 extends transversely across the ventilator 12 and connects with an opposite end of wall 20. Similarly, a second hinge 44 connects to an opposite end of wall 22. A rigid base member 43 can be provided which serves as anchor point for hinges 42 and 44, and also stabilizes the connection to valve 14. Hinges 42 and 44 can be of the conventional piano hinge type, or can be “living” hinges, which are simply thinned or narrower cross sectional delineations of material which naturally have a hinge action. Although a rigid base 43 is shown, it should also be understood that base 43 can be deleted in favor of a continuous pair of exterior walls 20 and 22 which are hinged at comers 45 and 46. Again at these corners, a living hinge or a conventional mechanical hinge could be provided.

The expiratory reservoir further includes an inlet 47, and a tube or connection 50 which connects with valve 14. The inspiratory reservoir includes an outlet 48 situated adjacent inlet 47, and a tube or connection 52 which connects with valve 14.

Now referring to FIG. 5, the specific construction of the valve 14 is shown. Valve 14 includes a body 60 which houses the internal components of the valve. A toggle/flapper 62 is secured at a hinge point 64 within the body 60 of the valve. The toggle 62 is biased to close off liquid flow from the outlet 48 of the inspiratory reservoir. The toggle 62 can be biased by a conventional spring (not shown) mounted at hinge point 64. Upon actuation of the ventilator causing liquid flow, the toggle 62 reverses its position to close off fluid flow from entering the expiratory reservoir through inlet 47. Accordingly, the toggle 62 has two distinct positions: (1)closing off access to the inspiratory reservoir at outlet 48, or (2)closing off access to the expiratory reservoir at inlet 47. The directional arrows A are provided to illustrate the direction of fluid flow through the valve and through the endotracheal tube, depending upon the expiration or inspiration phase of the patient. Inspiration arrows are denoted by A₁, and expiration is denoted by arrows A₂. The valve 14 and ventilator 12 could comprise an integral unit, or could be separate units which are connected prior to use via lines 50 and 52.

Now referring to FIG. 6, once the patient has been transported to an emergency room or other area for stabilization of the patient, the ventilator 12 can be mechanically actuated. As shown, a simple clamping device 70 may be used to hold the ventilator 12. Preferably the clamp 70 secures the rigid base 43. The mechanical actuating device can include a pair of opposing pistons 72 and 74 which make timed, simultaneous, and repetitive contact with respective exterior walls 20 and 22. Pistons 72 and 74 can be mounted within respective fluid cylinders 76 and 78. The pistons 72 and 74 can be actuated by hydraulic or pneumatic flow emanating from a pump (not shown) or any other well-known fluid forcing device.

The operation of the device will now be explained. Once the patient has received the endotracheal tube 18, tube 18 may be connected to valve 14. A predetermined amount of a first volume of perfluorocarbon can be pre-charged in the inspiratory reservoir immediately prior to use of the ventilator. The ventilator can be pre-charged through connection with the inlet or outlet of the inspiratory reservoir. The oxygenator/heat exchanger 16 would then be connected to the ventilator 12 via lines 34 and 36. A second volume of perfluorocarbon could be pre-charged in the oxygenator/chiller 16. Ventilation would commence by compressing exterior walls 20 and 22 to the position shown in FIG. 4. Upon compression, the toggle 62 would flip to close off access to the expiratory reservoir at inlet 47 thus opening the outlet 48 of the inspiratory reservoir allowing fluid flow of the first volume of perfluorocarbon fluid within the inspiratory reservoir to pass from the inspiratory reservoir, through valve 14, and into the patient's lungs (active inspiration) via endotracheal tube 18. In order to allow lung expiration, the compression force on exterior walls 20 and 22 would be released. Biased actuating means 38 and 40 would then force the walls 20 and 22 to their normally open position. As the volume within the expiratory reservoir expanded, this would create a suction force causing evacuation of fluid from the patient's lungs (active expiration) and passing the fluid into the expiratory reservoir. Simultaneous with evacuation of fluid from the patient's lungs, the second volume of perfluorocarbon within the oxygenator/heat exchanger 16 would empty into the inspiratory reservoir due to the suction force generated by the expanding volume of the inspiratory reservoir as wall 22 moved to its normally open position. Because fluid flow through the inspiratory reservoir would have ceased, toggle 62 would flip back to its normal position to close off the outlet 48 at the inspiratory reservoir. Accordingly, the expiratory reservoir would now contain the first volume of heated oxygen depleted perfluorocarbon from the patient's lungs, and the inspiratory reservoir would contain the second volume of cooled and oxygenated perfluorocarbon. The next inspiratory phase would commence by again providing a compression force to move the walls 20 and 22 towards their compressed positions. Once the walls 20 and 22 are compressed, the toggle would again flip to close off inlet 47 which would allow the contents of the inspiratory reservoir, namely, the second volume of oxygenated and recooled perfluorocarbon, to empty into the patient's lungs. The foregoing process is repeated as needed to provide liquid ventilation and pulmonary cooling.

From the foregoing, the advantages of the present invention are clear. A simple yet effective dual-chambered, active inspiration and active expiration ventilator is provided in a respiratory system or apparatus for liquid ventilation and pulmonary cooling. The present invention has utility both for a combination of elements, as well as a subcombination as to the ventilator. The ventilator is of a simple, yet reliable construction. The ventilator is portable, disposable, and can be either hand or mechanically actuated. Emergency medical personnel can use the ventilator for immediate liquid ventilation and pulmonary cooling to address the deleterious effects of cardiac arrest in a more timely manner.

This invention has been described in detail with reference to a particular embodiment thereof, but it will be understood that various other modifications can be effected within the spirit and scope of this invention.

What is claimed is:

1. A respiratory apparatus for liquid ventilation of a patient, said apparatus comprising:
first and second exterior movable walls;
an internal wall separating said first and second exterior walls, said interior wall having a first side facing said first exterior wall, and said interior wall having a second side facing said second exterior wall, said first exterior wall and said first side of said interior wall defining an expiratory reservoir and said second exterior wall and said second side of said interior wall defining an inspiratory reservoir;
a first biased actuating means communicating with said first exterior wall, and a second biased actuating means communicating with said second exterior wall;
said expiratory reservoir having an inlet and an outlet, said inlet and outlet of said expiratory reservoir being mounted on substantially opposing ends of said expiratory reservoir;
said inspiratory reservoir having an inlet and outlet, said inspiratory reservoir inlet and outlet being located at substantially opposing ends of said inspiratory reservoir, said inlet said outlet of said inspiratory reservoir and closing said inlet of said expiratory reservoir;
an oxygenator/heat exchanger connected between said outlet of said expiratory reservoir and said inlet of said inspiratory reservoir for oxygenating and providing temperature control of fluidic flow therethrough, said oxygenator/heat exchanger including an inlet for receiving oxygen depleted fluid from said expiratory reservoir, and including an outlet for delivering re-oxygenated and temperature controlled fluid to said inspiratory reservoir; and
an endotracheal tube communicating with flow of liquid through said valve, said endotracheal tube enabling fluidic flow to and from a patient's lungs; and
wherein a predetermined volume of liquid is circulated through said ventilator, said oxygenator/heat exchanger, and said endotracheal tube, said first and second exterior walls being actuated to simulate inspiration and expiration whereby the patient involuntarily respires in response as liquid is introduced into and withdrawn from said lungs.

2. An apparatus, as claimed in claim 1, further including:
hinge means connected to said first and second exterior walls enabling said first and second walls to be selectively rotated about said hinge means for inducing liquid flow through said ventilator.

3. An apparatus, as claimed in claim 1, further including:
a first check valve placed in line between said outlet of said expiratory reservoir and said inlet of said oxygenator/heat exchanger, and a second check valve placed in line between said inlet of said inspiratory reservoir and said outlet of said oxygenator/heat exchanger, said check valves preventing back flow of liquid through said ventilator.

4. An apparatus, as claimed in claim 1, further including:
means for mechanically actuating said first and second exterior walls, said mechanical actuating means providing timed actuation of said first and second exterior walls to match a desired respiration rate of the patient.

5. A respiratory apparatus for liquid ventilation of a patient, said apparatus comprising:
a ventilator including an expiratory reservoir having an inlet and an outlet, an inspiratory reservoir mounted adjacent said expiratory reservoir, said inspiratory reservoir including an inlet and an outlet and said ventilator further including means for adjusting the volume of said expiratory reservoir and said inspiratory reservoir in response to a desired respiration of a patient;
an oxygenator/heat exchanger mounted between said outlet of said expiratory reservoir and said inlet of said inspiratory reservoir, said oxygenator/heat exchanger for oxygenating liquid flowing through said ventilator and providing temperature control for said liquid;
a valve mounted in communication with said inlet of said expiratory reservoir and said outlet of said inspiratory reservoir, said valve for controlling flow of liquid from said inspiratory reservoir to lungs of a patient, and controlling flow of liquid from the patient's lungs to the inlet of said expiratory reservoir; and
an endotracheal tube communicating with said valve enabling the liquid to be introduced into and withdrawn from the patient's lungs.

6. An apparatus, as claimed in claim 5, further including:
a first check valve communicating with said outlet of said expiratory reservoir, and a second check valve communicating with said inlet of said inspiratory reservoir, said first and second check valves preventing back flow of liquid.

7. An apparatus, as claimed in claim 5, further including:
means for mechanically actuating said ventilator, said mechanical actuating means providing timed actuation to simulate a desired respiration rate of the patient.

8. In subcombination, a ventilator especially adapted for use in a respiratory apparatus for liquid ventilation, said ventilator comprising:
first and second exterior movable walls;
an internal wall separating said first and second exterior walls, said interior wall having a first side facing said first exterior wall, and said interior wall having a second side facing said second exterior wall, said first exterior wall and said first side of said interior wall defining an expiratory reservoir and said second exterior wall and said second side of said interior wall defining an inspiratory reservoir;
a first biased actuating means communicating with said first exterior wall, and a second biased actuating means communicating with said second exterior wall;
said expiratory reservoir having an inlet and an outlet, said inlet and outlet of said expiratory reservoir being mounted on substantially opposing ends of said expiratory reservoir;
said inspiratory reservoir having an inlet and outlet, said inspiratory reservoir inlet and outlet being located at substantially opposing ends of said inspiratory reservoir, said inlet of said expiratory reservoir and said outlet of said inspiratory reservoir being placed adjacent one another at one end of said ventilator;

9. In subcombination, a ventilator especially adapted for use in a respiratory apparatus for liquid ventilation of a patient, said ventilator comprising:
an expiratory reservoir having an inlet and on outlet;
an inspiratory reservoir mounted adjacent said expiratory reservoir and having an inlet and an outlet;
means for adjusting respective volumes of said expiratory reservoir and said inspiratory reservoir in response to a desired respiration rate of a patient, wherein adjustment of said volumes occurs simultaneously resulting in a fluid flow through said ventilator to deliver fluid to the patient during inspiration and to withdraw fluid from the patient's lungs during expiration.

10. A respiratory apparatus for liquid ventilation of a patient, said apparatus comprising:
first and second exterior movable walls;
mean for separating said first and second exterior walls, said means for separating having a first side facing said first exterior wall, and said means for separating having a second side facing said second exterior wall, said first exterior wall and said first side of said means for separating defining an expiratory reservoir and said second exterior wall and said second side of said means for separating defining an inspiratory reservoir;
means for actuating said first and second exterior walls urging said first and second walls in an open position
said expiratory reservoir having an inlet and an outlet;
said inspiratory reservoir having an inlet and outlet;
an oxygenator/heat exchanger connected between said outlet of said expiratory reservoir and said inlet of said inspiratory reservoir for oxygenating and providing temperature control of fluidic flow therethrough, said oxygenator/heat exchanger including an inlet for receiving oxygen depleted fluid from said expiratory reservoir, and including an outlet for delivering re-oxygenated and temperature controlled fluid to said inspiratory reservoir; and
an endotracheal tube communicating with flow of liquid through said valve, said endotracheal tube enabling fluidic flow to and from a patient's lungs; and
wherein a predetermined volume of liquid is circulated through said ventilator, said oxygenator/heat exchanger, and said endotracheal tube, said first and second exterior walls being actuated to simulate inspiration and expiration whereby the patient involuntarily respires in response as liquid is introduced into and withdrawn from said lungs.
